(19) United States
(12) Patent Application Publication
(10) Pub. No.: US 2026/0054035 A1
(43) Pub. Date: Feb. 26, 2026

(54) CATHETER ASSEMBLY

(71) Applicant: Becton, Dickinson and Company, Franklin Lakes, NJ (US)

(72) Inventors: Sandarsha Shetty, Bengaluru (IN); Ravikumar Mohan, Chennai (IN); Suraj Govindaraju, Tumkur (IN)

(73) Assignee: Becton, Dickinson and Company, Franklin Lakes, NJ (US)

(21) Appl. No.: 18/814,454

(22) Filed: Aug. 23, 2024

(51) Int. Cl. A61M 25/06; A61M 25/00; A61M 39/22; A61M 39/24

(52) U.S. Cl. CPC A61M 25/0606; A61M 25/0097; A61M 39/22; A61M 2039/2426; A61M 2205/0216; A61M 2205/025

(57) ABSTRACT

A catheter assembly comprising a catheter and a catheter hub connected to the catheter. The catheter hub including a septum valve that is configured to permit or block a flow of fluid through the catheter, a valve actuator movable between a first position and a second position, the valve actuator opening the septum valve in the second position to permit the flow of fluid therethrough, an actuator ring fixed to the catheter hub, and an elastomeric bellow member attached to the actuator ring and the valve actuator. The elastomeric bellow member is configured to expand when the valve actuator moves from the first position where the septum valve is closed to the second position where the septum valve is open, and the elastomeric bellow member is configured to retract to cause the valve actuator to retract from the second position to the first position.

### FIELD

Various exemplary embodiments of the invention relate to catheter assemblies.

### BACKGROUND

Catheter assemblies are used to place a catheter properly into the vascular system of a patient. Once in place, catheters such as intravenous catheters may be used to infuse fluids including normal saline, medicinal compounds, and/or nutritional compositions into a patient in need of such treatment. Catheters additionally enable the removal of fluids from the circulatory system and monitoring of conditions within the vascular system of the patient.

Catheter assemblies are often used in and during a magnetic resonance imaging (MRI) procedure. Recent trends indicate that the power of MRI machines will increase over time. Currently, in the U.S., the FDA has limited the power of MRI procedures for clinical use to 8T (8 Tesla), while Europe (IEC) has limited the power of MRI procedures to 4T (4 Tesla). That being said, 7T and 11T MRI tests are being conducted and Europe tends to follow the lead of the U.S.

The following publications disclose elevated use of power in MRI procedures. Chang, Eric “Morphologic characterization of meniscal root ligaments in the human knee with magnetic resonance microscopy at 11.7 and 3T” Skeletal Radiol No. 43 (June 2014) pages 1395-1402. Hoff, Michael “Safety Considerations of 7-T MRI in Clinical Practice” Radiology: Volume 292: Number 3 (September 2019) pages 509-518. radiology.rsna.org. van Osch, M. J. P., Webb, A. G. Safety of Ultra-High Field MRI: What are the Specific Risks?. Curr Radiol Rep 2, 61 (2014). https://doi.org/10.1007/s40134-014-0061-0. Thus, there is a need for improved metal sensitive catheter assemblies for procedures including MRI, sterilization and others.

### 1. SUMMARY OF THE INVENTION

It is an aspect of the present invention to provide a catheter assembly having a refined actuator design to achieve a MRI-safe multi-guard technology. This refined actuator design includes a polymeric actuator retainer and a spring back movement via a polymeric disc. This refined actuator design replaces a metal wedge used to assemble a catheter tube in a catheter adapter and also replaces a metal spring for actuation of a valve actuator. The use of plastic and polymer parts instead of metal parts makes the catheter assembly safer during MRI procedures. Accordingly, these catheter assembly improvements contribute to patient comfort by eliminating the need for additional peripheral intravenous catheter (PIVC) changes during an MRI procedure.

Specifically, the polymeric actuator retainer includes an actuator ring fixed to the catheter adapter, and an elastomeric bellow member attached to the actuator ring and the valve actuator. In this configuration, the elastomeric bellow member is configured to expand when the valve actuator moves from a first position where the septum valve is closed to a second position where the septum valve is open, and the elastomeric bellow member is configured to retract to cause the valve actuator to retract from the second position to the first position.

Additionally, the polymeric disc includes a disc spring disposed distal to the septum valve. The disc spring is adjacent to and contacts a distal surface of the septum valve. The polymeric spring aids in retracting the valve actuator from the second position where the septum valve is open, to the first position. The polymeric spring also aids to close the septum when the valve actuator is moved from the second position to the first position.

Accordingly, it is an aspect of the present invention to provide a catheter assembly comprising a catheter and a catheter hub connected to the catheter. The catheter hub including a septum valve that is configured to permit or block a flow of fluid through the catheter, a valve actuator movable between a first position and a second position, the valve actuator opening the septum valve in the second position to permit the flow of fluid therethrough, an actuator ring fixed to the catheter hub, and an elastomeric bellow member attached to the actuator ring and the valve actuator. The elastomeric bellow member is configured to expand when the valve actuator moves from the first position where the septum valve is closed to the second position where the septum valve is open, and the elastomeric bellow member is configured to retract to cause the valve actuator to retract from the second position to the first position.

Another aspect of the present invention is to provide a catheter assembly comprising a catheter and a catheter hub connected to the catheter. The catheter hub including a septum valve that is configured to permit or block a flow of fluid through the catheter, a valve actuator movable between a first position and a second position, the valve actuator opening the septum valve in the second position to permit the flow of fluid therethrough, and a disc spring contacting the septum valve to close the septum valve.

Additional and/or other aspects and advantages of the present invention will be set forth in the description that follows, or will be apparent from the description, or may be learned by practice of the invention.

### 2. BRIEF DESCRIPTION OF THE DRAWINGS

The above aspects and features of the present invention will be more apparent from the description for the exemplary embodiments of the present invention taken with reference to the accompanying drawings, in which:

FIG. 1 is an exploded perspective view of a related art catheter assembly;

FIG. 2 is a flow diagram illustrating a method of using the catheter assembly of FIG. 1;

FIG. 3 is an exploded perspective view of an exemplary catheter assembly;

FIG. 4 is a transparent, perspective view of the catheter assembly of FIG. 3;

FIG. 5 is a cross-sectional view of the catheter assembly of FIG. 3 when the septum valve is open;

FIG. 6 is a perspective view of an elastomeric bellow member in the catheter assembly of FIG. 3;

FIG. 7 is a perspective view of a disc spring in the catheter assembly of FIG. 3;

FIG. 8 is a cross-sectional view of the catheter assembly of FIG. 3 when the septum valve is closed;

FIG. 9 is a cross-sectional view of an exemplary valve actuator assembly; and

FIG. 10 is a cross-sectional view of another exemplary valve actuator assembly.

### DETAILED DESCRIPTION OF EXEMPLARY EMBODIMENTS

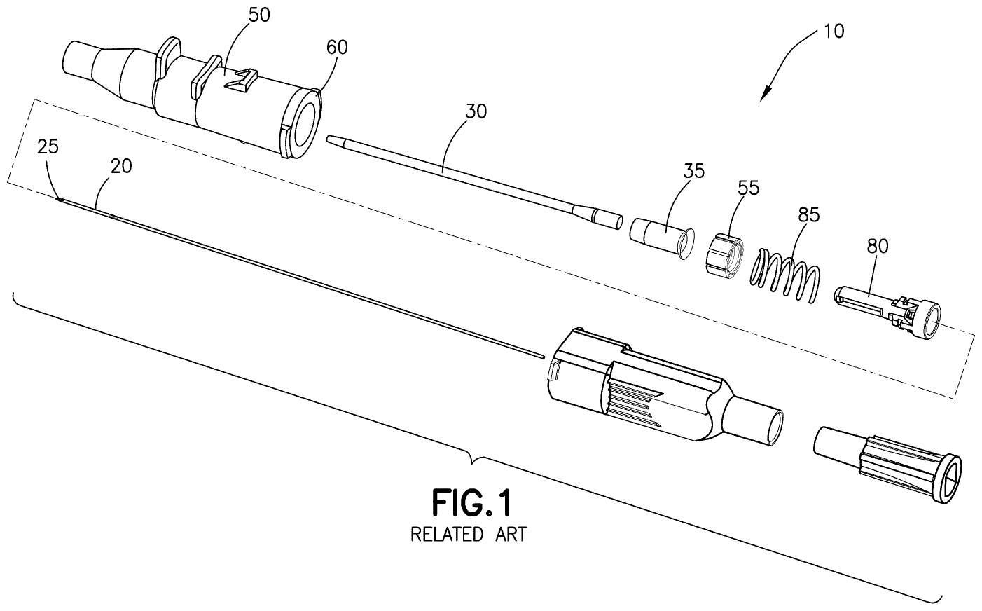

A catheter assembly 10, as understood by one skilled in the art and as shown in FIG. 1, is referred to as multi-guard technology and includes a hollow introducer needle 20, a catheter tube 30, a metal wedge 35, a catheter hub 50, a septum valve 55, a luer adapter 60, a valve actuator 80 and a spring or return member 85. The introducer needle 20 has a sharpened distal end 25 and extends through the catheter hub 50. A flexible catheter tube 30 extends from a distal end of the catheter hub 50, with the needle 20 passing through the catheter tube 30. A similar catheter assembly is disclosed in U.S. Pat. No. 10,960,186 which is incorporated herein by reference. Multi-guard technology eliminates the need for venous compression during subsequent connections and disconnections while advantageously reducing a clinician's exposure to blood borne pathogens.

The metal wedge 35 may be positioned in a channel of the catheter hub 50 to secure the catheter tube 30 in the catheter opening. The wedge 35 has a first end engaging the catheter tube 30 and a second end engaging an inner diameter of the catheter hub 50. A pre-slit resilient septum valve 55 is positioned in the catheter hub 50 and functions as a septum valve that forms a fluid-tight seal and selectively admits fluid to or from the flexible catheter tube 30. In other words, the septum valve 55 is configured to permit or block the flow of fluid through the flexible catheter tube 30. The septum valve 55 is fixed to the inner diameter of the catheter hub 50 via adhesive or a frictional press fit.

The valve actuator 80 is positioned in the catheter hub 50 and is axially moveable in the catheter hub 50 to open and close slits of the septum valve 55. When the septum valve 55 is closed, the valve actuator 80 is in a first position. As a male Luer connector is inserted in the catheter hub 50, the end of the male Luer connector engages the luer adapter 60 disposed at a proximal end of the catheter hub 50, and abuts the valve actuator 80 (not illustrated but understood by one skilled in the art). Further movement of the Luer connector moves the valve actuator 80 axially toward and through the septum valve 55 with a distal end of the valve actuator 80 separating the one or more slits of the septum valve 55 to engage and open the septum valve 55. Without the activation from the male Luer connector, the weight of the valve actuator 80 is unable to penetrate the septum valve 55 and a polymeric disc spring 240 (see below).

At this point, the valve actuator 80 is in a second position where the septum valve 55 is open. After the septum valve 55 is opened by the valve actuator 80, fluid is permitted to flow from the Luer connector, through internal passages of the valve actuator 80, and into the flexible catheter 30 or vice versa. When the Luer connector is removed, the valve actuator 80 disengages the septum valve 55 and returns the valve actuator 80 to its initial position (or the first position) via the return member 85.

The return member 85 includes a biasing member such as a coil spring, or a helical compression spring. The spring can be, but is not limited to, metal, a biocompatible metal or the like. The return member 85 can also be a resilient septum valve 55 via the slits or flaps of the septum valve 55 forcing the valve actuator 80 to return from the second position to the first position. The return member 85 functions by returning the valve actuator 80 from the second position engaging the septum valve 55 (opening or penetrating the septum, for example) to open the septum, to the first position at a proximal end of the septum valve 55 (not engaging the septum valve 55) to close the septum.

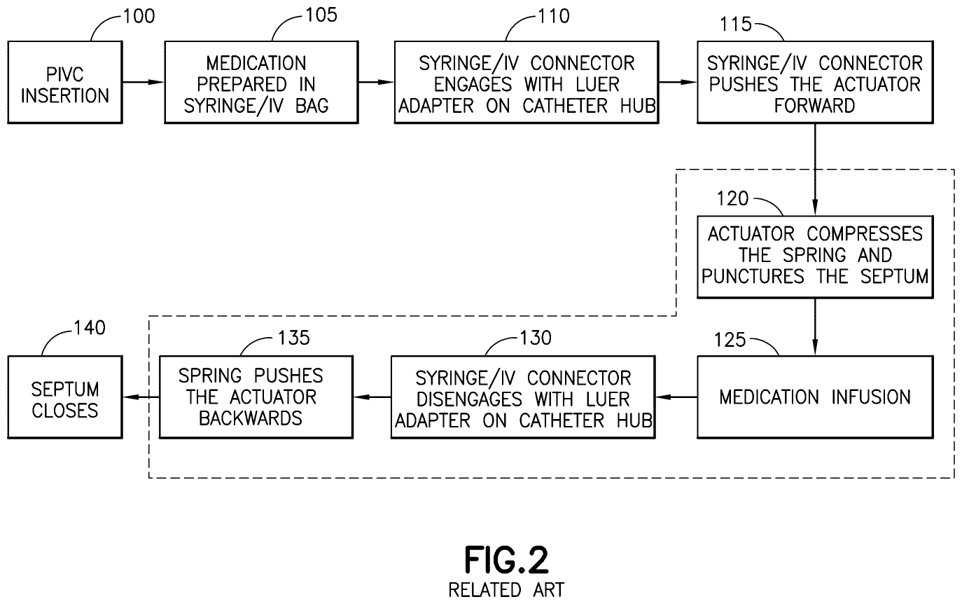

FIG. 2 shows a flow diagram illustrating the operation of the catheter assembly of FIG. 1. Initially, the needle 20 and the catheter tube 30 are inserted into a patient's vein. In other words, a peripheral intravenous catheter (PIVC) is the catheter tube 30 inserted into the vein using the needle 20 (step 100). After the catheter tube 30 is inserted, the needle 20 is removed from the patient's vein and the catheter hub 50, leaving the catheter tube 30 inside the patient as the needle 20 is discarded.

Preferably, during or after catheter tube 30 insertion, medication is prepared in a syringe or an intravenous (IV) bag (step 105). In step 110, the syringe or intravenous connector of the IV bag engages with the luer adapter 60 on the catheter hub 50. Next, in step 115, a user pushes the syringe or intravenous connector to move the valve actuator 80 toward the septum valve 55. In step 120, the valve actuator 80 compresses the spring or return member 85 and punctures the septum valve 55 to open the septum valve 55 and establish fluid communication.

In step 125, medication infusion is administered by medication flowing from the syringe or IV bag and into the vein of the patient. Blood can also be removed from the vein of the patient in the opposite fluid flow direction with respect to medication infusion. In step 130, the syringe/intravenous connector disengages from the luer adapter 60 on the catheter hub 50. This disengagement causes the spring or return member 85 to push the valve actuator 80 backwards to its original position as set forth in step 135. Finally, as a result in step 140, the septum valve 55 closes.

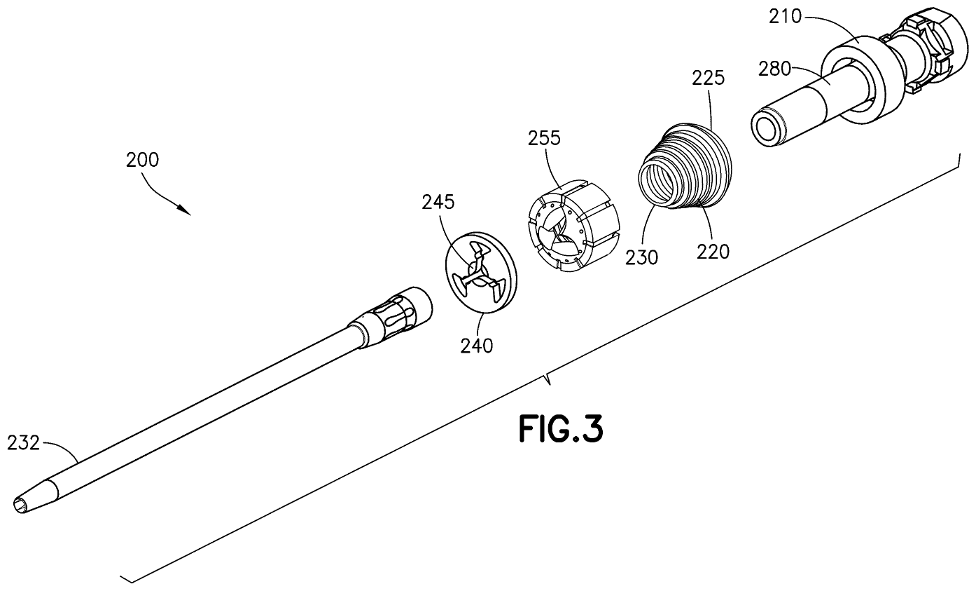

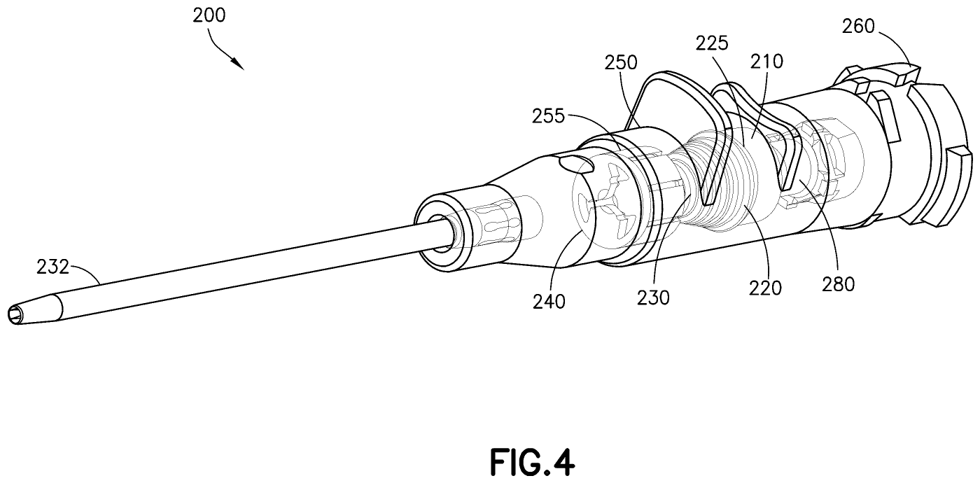

FIGS. 3 and 4 illustrate an exemplary embodiment of the catheter assembly 200 in accordance with an embodiment of the present invention. The catheter assembly 200 is similar to the catheter assembly 10 described above except for the following differences. The catheter assembly 200 includes an actuator ring 210, an elastomeric bellow member 220, and a polymeric disc spring 240. Specifically, the metal spring 85 of the catheter assembly 10 is replaced with these three components of the catheter assembly 200. Moreover, the metal wedge 35 of the catheter assembly 10 is replaced by a plastic wedge 235 (see FIGS. 5 and 8, for example) in the catheter assembly 200. These differences advantageously provide a refined actuator design to achieve the MRI-safe catheter experience.

The actuator ring 210 comprises a hollow ring element fixed at an inner diameter of the catheter hub 250 via an interference fit. The actuator ring 210 is preferably an elastomeric member with a high rate of elasticity but can also be a variety of other MRI compatible materials. More preferably, the actuator ring 210 is composed of a thermoset silicone. However, other polymers such as thermoplastic elastomers (TPEs) and thermoplastic polyurethane (TPUs) can be employed.

As illustrated in FIG. 5, the actuator ring 210 is disposed centrally between the septum valve 255 and the luer adapter 260 to advantageously allow the valve actuator 280 to travel therethrough and cooperate with the elastomeric bellow member 220 as further described below. Although FIG. 3 appears to illustrate the actuator 210 and the valve actuator 280 as a single part, these two components are separate elements as illustrated in FIG. 5, and cooperate together as disclosed herein.

The elastomeric bellow member 220 is hollow and, as illustrated in FIGS. 4-6, includes a lateral opening 222, a proximal end 225, a flange 227 and a distal end 230. A hole extends through the proximal end 225 to the distal end 230 of the elastomeric bellow member 220 to create a hollow cavity. The elastomeric bellow member 220 includes a thin radial wall that generally decreases in outer diameter as it extends from the flange 227 to the distal end 230. An outer diameter of the flange 227 is slightly smaller than the inner diameter of the catheter hub 250 where the elastomeric bellow member 220 is positioned. The proximal end 225 of the elastomeric bellow member 220 includes an outer diameter smaller than the outer diameter of the flange 227 and is fixed to a distal end of the actuator ring 210. The distal end 230 of the elastomeric bellow member 220 includes an inner diameter slightly larger than the outer diameter of the valve actuator 280 and is fixed to the valve actuator 280. The proximal end 225 and the distal end 230 of the elastomeric bellow member 220 are fixed to the actuator ring 210 and the valve actuator 280, respectively, via glue, overmolding, or are assembled through a polymer welding method.

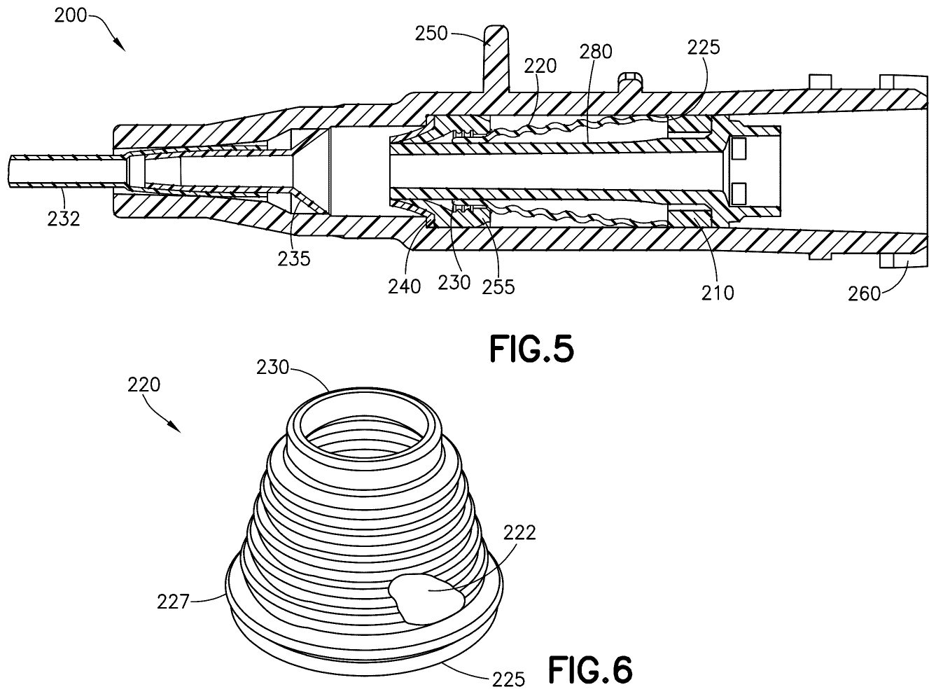

The elastic force of the elastomeric bellow member 220 is a function of the modulus of the material, the wall thickness and length of the elastomeric bellow member 220. Additionally, the elastomeric bellow member 220 includes the lateral opening 222 to adjust the elastic force properties for suitable operation in the catheter assembly 200. The lateral opening 222 is distally disposed with respect to the flange 227 and disposed asymmetrically with respect to a centerline of the elastomeric bellow member 220. FIG. 6 exaggerates the dimensions of the lateral opening 222 for emphasis purposes only. In operation, the lateral opening 222 are about 0.5 mm in diameter.

The lateral opening 222 advantageously prevents a formation of a vacuum/suction during activation of the valve actuator 280. Specifically, the lateral opening 222 prevents compressed air from being retained inside a space between the elastomer bellow member 220 and the valve actuator 280. If the compressed air was retained, over time this configuration could prevent the valve actuator 280 from retracting completely to its detriment.

As a result of the configuration described above, the valve actuator 280 is advantageously constrained in the catheter assembly 200 via the elastomeric bellow member 220 to prevent the valve actuator 280 from falling out. The flange 227 is sized to be slightly smaller than the inner diameter of the catheter hub 250 to advantageously center and radially position the elastomeric bellow member 220 for optimal operation and life. As a result, the flange 227 will be appropriately constrained within the inner diameter of the catheter hub 250 and adjacent to the actuator ring 210. The elastomeric bellow member 220 is preferably composed of polydimethyl siloxane. However, other elastomers such as thermoplastic polyurethane, thermoplastics vulcanazates, fluoroclastomers, low compression set radial triblock elastomers, isoprene, and other biocompatible low compression set elastomers with sufficient modulus can also be employed.

In another embodiment, the catheter assembly 200 does not include the actuator ring 210. Instead, the elastomeric bellow member 220 is fixed directly to the inner diameter of the catheter hub 250 via glue, overmolding, or is assembled through a polymer welding method (not illustrated).

FIG. 4 illustrates the valve actuator 280 is in a first position where the septum valve 255 is closed, and is not pierced. In this position, the elastomeric bellow member 220 is in a rested position, or a non-elongated position. By virtue of the modulus of elasticity of the material of the elastomeric bellow member 220, the valve actuator 280 is held in place and the distal end of the valve actuator 280 is resting on the septum valve 255 with a negligible normal force applied on the septum valve 255. Accordingly, no force acts upon the elastomeric bellow member 220 in the first position of the valve actuator 280.

FIG. 5 illustrates the valve actuator 280 beginning to travel to a second position where the septum valve 255 is pierced and opened. FIG. 5 shows the septum valve 255 open with the valve actuator 280 in the second position. In the second position of the valve actuator 280, the elastomeric bellow member 220 stretches or elongates with the movement of the valve actuator 280 and ultimately acts as a bellow spring in tension to ultimately return the valve actuator 280 to the first position.

The fluid communication is complete between the catheter tube 230 and the catheter hub 250 when, for example, the male Luer connector is unplugged from the luer adapter 260. When the male Luer connector is removed, the end of the male Luer connector no longer contacts the valve actuator 280. When this takes place, the valve actuator 280 returns to the first position and the septum valve 255 is closed. This is because the elastomeric bellow member 220, when the valve actuator 280 is in the second position, is biased to return to the rest state due to its elastic nature. Thus, the elastomeric bellow member 220 causes the valve actuator 280 to retract from the second position to the first position.

Elastomeric springs such as the elastomeric bellow member 220 tend to creep when a constant load is applied for a prolonged period, such as during storage or continuous loading during operation. This may lead to a permanent creep flow or plastic deformation, which may prevent the valve actuator 280 from retracting completely. These challenges are advantageously minimized in the catheter assembly 200 because of the use of the polymeric disc spring 240 as further described below.

FIGS. 4, 5, and 7 illustrate the polymeric disc spring 240 in the catheter assembly 200. The material of the polymeric disc spring 240 has sufficient modulus and flexure bearing capacity to close the septum valve 255 and move the valve actuator 280 from the second position to the first position. According to one exemplary embodiment, the polymeric disc spring 240 is a leaf spring comprised of one of a variety of MRI compatible material such as a polymer. Preferably, the material employed for the polymeric disc spring 240 is a polypropylene elastomer with polypropylene being the dominant matrix and ethylene propylene diene monomer (EPDM) or poly [styrene-b-(ethylene-co-butylene)-b-styrene] (SEBS) or any other triblock polymers as the secondary soft component. Other polyolefins such as polyethylene, and polybutylene can also be substituted in place of polypropylene. Other possible materials may include, but are not limited to, polyamides, thermoplastic copolyester elastomer, fluoroelastomers, crosslinked polyurethanes and polyimide elastomers.

The polymeric disc spring 240 acts as a support to the valve actuator 280, such that the polymeric disc spring 240 reduces the elongational load on the elastomeric bellow member 220 when the polymeric disc spring 240 is not activated. In other words, the polymeric disc spring 240 provide resistance against the elongation of the elastomeric bellow member 220 along a central axis.

Further, the polymeric disc spring 240 has a higher modulus of elasticity than the elastomeric bellow member 220 to advantageously unload the polymeric disc spring 240 when the septum valve 255 is closed. Further, a higher modulus of elasticity ensures appropriate spring back when the septum valve 255 is opened. Thus, the greater stiffness of the polymeric disc spring 240, when compared to the elastomeric bellow member 220, advantageously provides an adequate secondary force to move the valve actuator 280 from the second position to the first position. This configuration advantageously also further extends the life of both the polymeric disc spring 240 and the elastomeric bellow member 220.

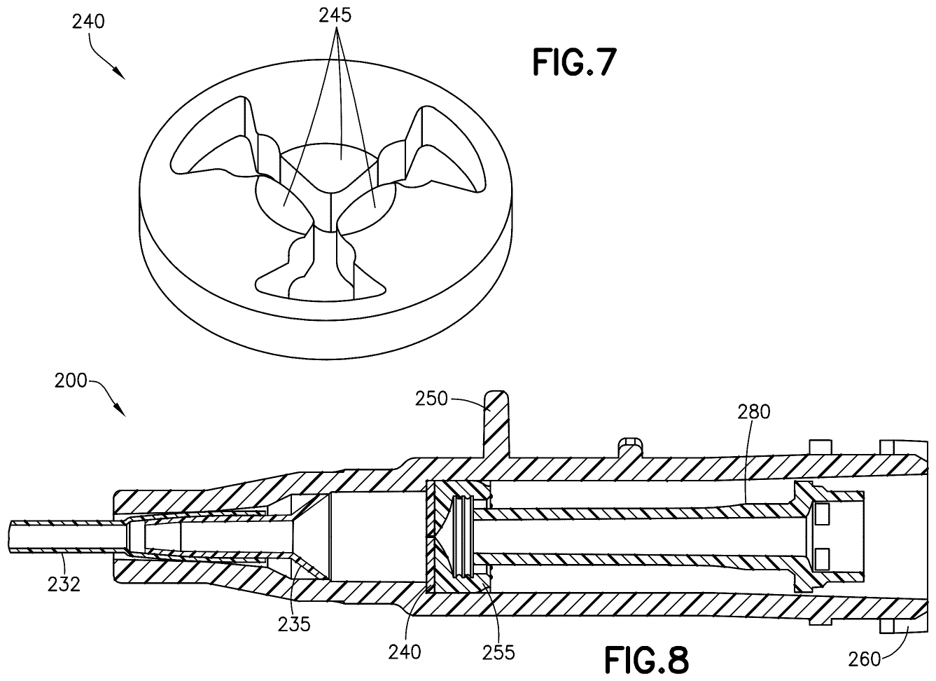

As illustrated in FIG. 7, the polymeric disc spring 240 includes a plurality of flaps 245 or leaves that are cantilevered and deflect to allow the valve actuator 280 to extend through in the second position. FIG. 7 illustrates three flaps 245, although more or fewer are contemplated, and are within the scope of the present invention. According to one embodiment, the polymeric disc spring 240 is disposed distal to the septum valve 255. The disc spring 240 is also adjacent to and contacts a distal surface of the septum valve 255. This contact provides secondary resilience to close the septum valve 255 (secondary to the elastomeric bellow member 220) and ultimately move the valve actuator 280 from the second position to the first position.

When the connector is unplugged from the luer adapter 260, as described above, the end of the male Luer connector no longer contacts the valve actuator 280. Accordingly, the elastomeric bellow member 220 in combination with disc spring 240 moves the valve actuator 280 from the second position to the first position while closing the septum valve 255. The flaps 245 of the polymeric disc spring 240 tend to spring back to their original shape due to their resilient, clastic nature. Thus, the spring back action forces the septum valve 255 to close with a tight seal while simultaneously assisting the valve actuator 280 to move from the second position to the first position. That is, the polymeric disc spring 240 desires to return to its natural, unloaded state to coincide with the closure of the septum valve 255.

As the valve actuator 280 moves from the second position to the first position, the valve actuator 280 does not come in direct contact with the polymeric disc spring 240. Nevertheless, the polymeric disc spring 240 indirectly supports the valve actuator 280 and the elastomeric bellow member 220 in the second positon, and during a portion of the transition when the valve actuator 280 moves from the second position to the first position. During this transition, the polymeric disc spring 240 only comes in direct contact with the catheter hub 250 and the distal surface of the septum valve 255. Additionally, the geometry, travel and contact area of the septum valve 255 and the valve actuator 280, in cooperation with the polymeric disc spring 240, is advantageously designed to avoid friction preventing smooth operation between the polymeric disc spring 240 and the valve actuator 280. Thus, the spring back action of the polymeric disc spring 240 can advantageously supplement or reinforce the force from the elastomeric bellow member 220, as well as reduce any creep flow that may develop over time.

The combination of the actuator ring 210 with the elastomeric bellow member 220, as well as the polymeric disc spring 240 advantageously minimizes the risk of creep over the operational life of the catheter assembly 200. Also, and importantly, this combination of features advantageously provides spring back and proper operation of the catheter assembly 200 without the use of metal parts (without metallic components), thus achieving MRI-safe status and compatibility with other procedures and sterilizations. The catheter assembly 200 advantageously provides patient comfort by eliminating the need for additional PIVC change during an MRI or other medical procedure. Although the catheter assembly 200 includes additional parts, as well as additional manufacturing and assembly steps, the resulting benefits of the catheter assembly 200 provide advantages that distinguish from related art devices.

In another embodiment, the catheter assembly 200 includes the actuator ring 210 and the elastomeric bellow member 220 but not the polymeric disc spring 240. In yet another embodiment, as illustrated in FIG. 8, the catheter assembly 200 includes the polymeric disc spring 240 but not the elastomeric bellow member 220 and the actuator ring 210. The elastomeric bellow member 220 with the actuator ring 210, and the polymeric disc spring 240 provide unique and separate advantages that can be combined in the catheter assembly 200 but these features and their advantages can also be realized in separate and independent catheter assemblies.

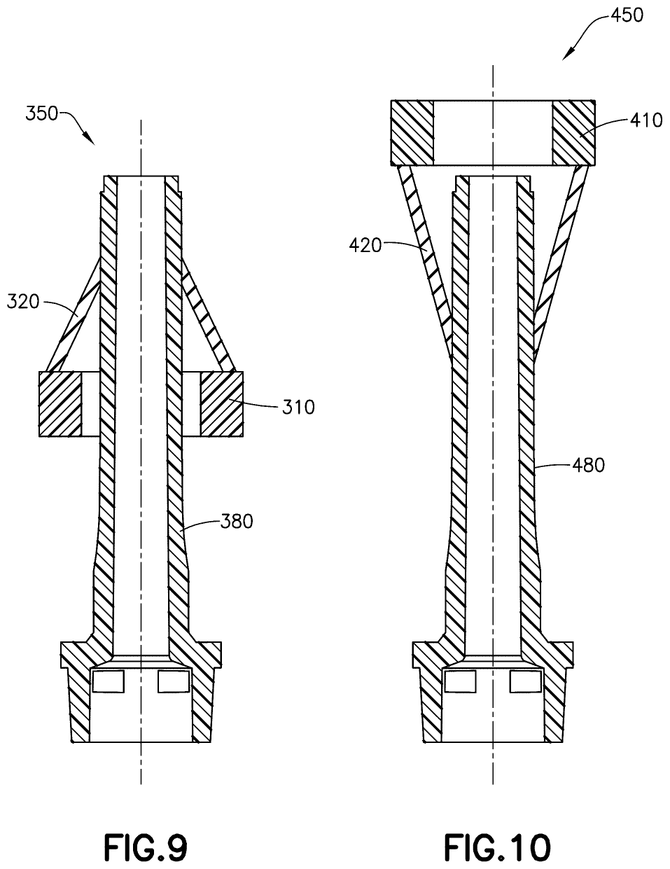

FIGS. 9 and 10 illustrate alternate embodiments of a valve actuator assembly 350, 450 in the catheter assembly 300, 400. Specifically, FIG. 9 illustrates the elastomeric bellow member being an elastic membrane 320 that is attached to the valve actuator 380 and the actuator ring 310 by glue or is assembled by a polymer welding method. Alternately, the elastic membrane 320 can be overmolded on one of the actuator ring 310 and the valve actuator 380, and the clastic membrane 320 can be glued or polymer welded on the other of the actuator ring 310 and the valve actuator 380. These embodiments advantageously provide alternate means of securing the actuator ring 310.

FIG. 10 illustrates an embodiment of the valve actuator assembly 450 in which the valve actuator 480 and the actuator ring 410 are both overmolded by the clastic membrane 420. This configuration advantageously allows the actuator ring 410 to be moved away from the luer connector side to provide another unique valve actuator 480 retraction configuration. Overmolding advantageously allows for the secure joining of two dissimilar polymers. FIG. 10 is a representative figure for the overmolding process. After the overmolding process is complete, the assembly will be refit to operate in the manner disclosed with respect to FIG. 9.

The foregoing detailed description of the certain exemplary in embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. Any of the embodiments and/or elements disclosed herein may be combined with one another to form various additional embodiments not specifically disclosed. Accordingly, additional embodiments are possible and are intended to be encompassed within this specification and the scope of the invention. The specification describes specific examples to accomplish a more general goal that may be accomplished in another way.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges outside of the given value, for example, general tolerances associated with manufacturing, assembly, and use of the described embodiments.

1. A catheter assembly comprising:
a catheter;
a catheter hub connected to the catheter, the catheter hub including:
a septum valve that is configured to permit or block a flow of fluid through the catheter;
a valve actuator movable between a first position and a second position, the valve actuator opening the septum valve in the second position to permit the flow of fluid therethrough;
an actuator ring fixed to the catheter hub; and
an elastomeric bellow member attached to the actuator ring and the valve actuator, wherein:
the elastomeric bellow member is configured to expand when the valve actuator moves from the first position where the septum valve is closed to the second position where the septum valve is open; and
the elastomeric bellow member is configured to retract to cause the valve actuator to retract from the second position to the first position.

2. The assembly of claim 1, wherein the actuator ring is fixed to an inner diameter of the catheter hub via an interference fit.

3. The assembly of claim 1, wherein the elastomeric bellow member comprises an elastic membrane.

4. The assembly of claim 1, wherein the elastomeric bellow member comprises polydimethyl siloxane.

5. The assembly of claim 1, wherein:
the elastomeric bellow member includes a proximal end and a distal end;
the proximal end of the elastomeric bellow member is attached to the actuator ring; and
the distal end of the elastomeric bellow member is attached to the valve actuator.

6. The assembly of claim 1, wherein the elastomeric bellow member is attached to the actuator ring and the valve actuator by at least one of a glue application and an overmolding procedure.

7. The assembly of claim 1, wherein the elastomeric bellow member is in a rest state to movably hold the valve actuator in the first position.

8. The assembly of claim 1, wherein the assembly does not include metallic components.

9. The assembly of claim 1, further comprising a disc spring that assists the elastomeric bellow member in moving the valve actuator from the second position to the first position.

10. The assembly of claim 9, wherein the disc spring comprises a polypropylene elastomer.

11. The assembly of claim 9, wherein the disc spring is disposed distal to the septum valve.

12. The assembly of claim 9, wherein the disc spring contacts the septum valve to close the septum valve.

13. The assembly of claim 11, wherein the disc spring includes a plurality of flaps that elastically move when the valve actuator opens the septum valve.

14. A catheter assembly comprising:
a catheter;
a catheter hub connected to the catheter, the catheter hub including:
a septum valve that is configured to permit or block a flow of fluid through the catheter;
a valve actuator movable between a first position and a second position, the valve actuator opening the septum valve in the second position to permit the flow of fluid therethrough; and
a disc spring contacting the septum valve to close the septum valve.

15. The assembly of claim 14, wherein the disc spring comprises a polypropylene elastomer.

16. The assembly of claim 14, wherein when a luer adapter of the catheter hub is disengaged, the valve actuator moves from the second position to the first position and the septum valve closes.

17. The assembly of claim 14, wherein when a luer adapter of the catheter hub is engaged, the valve actuator moves to the second position to open the septum valve and elastically open a plurality of flaps of the disc spring.
